(19) United States
(12) Patent Application Publication
(10) Pub. No.: US 2020/0085216 A1
(43) Pub. Date: Mar. 19, 2020

(54) HEATED PILLOWCASE ASSEMBLY

(71) Applicant: Amanda H. Anderson, Cherry Hill, NJ (US)

(72) Inventor: Amanda H. Anderson, Cherry Hill, NJ (US)

(21) Appl. No.: 16/129,969

(22) Filed: Sep. 13, 2018

(51) Int. Cl. A47G 9/10; A47G 9/02; A61F 7/00

(52) U.S. Cl. CPC A47G 9/1036; A47G 9/0253; A61H 2205/02; A61F 2007/0002; A61H 2201/0157; A61F 7/007

(57) ABSTRACT

A heated pillowcase assembly for keeping a user warm without a blanket includes a pillowcase that has a length of at least 50.0 inches and a width of at least 20.0 inches. A body pillow can be positioned in the pillowcase such that the pillowcase rests against a user when the user lies against the body pillow. A heating unit is coupled to the pillowcase and the heating unit is in thermal communication with the pillowcase. The heating unit heats the pillowcase when the heating unit is turned on to keep the user warm when the user lies against the body pillow.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to pillowcase devices and more particularly pertains to a new pillowcase device for keeping a user warm without a blanket.

### BRIEF SUMMARY OF THE INVENTION

An embodiment of the disclosure meets the needs presented above by generally comprising a pillowcase that has a length of at least 50.0 inches and a width of at least 20.0 inches. A body pillow can be positioned in the pillowcase such that the pillowcase rests against a user when the user lies against the body pillow. A heating unit is coupled to the pillowcase and the heating unit is in thermal communication with the pillowcase. The heating unit heats the pillowcase when the heating unit is turned on to keep the user warm when the user lies against the body pillow.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a heated pillowcase assembly according to an embodiment of the disclosure.

FIG. 2 is a bottom phantom view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

### DETAILED DESCRIPTION OF THE INVENTION

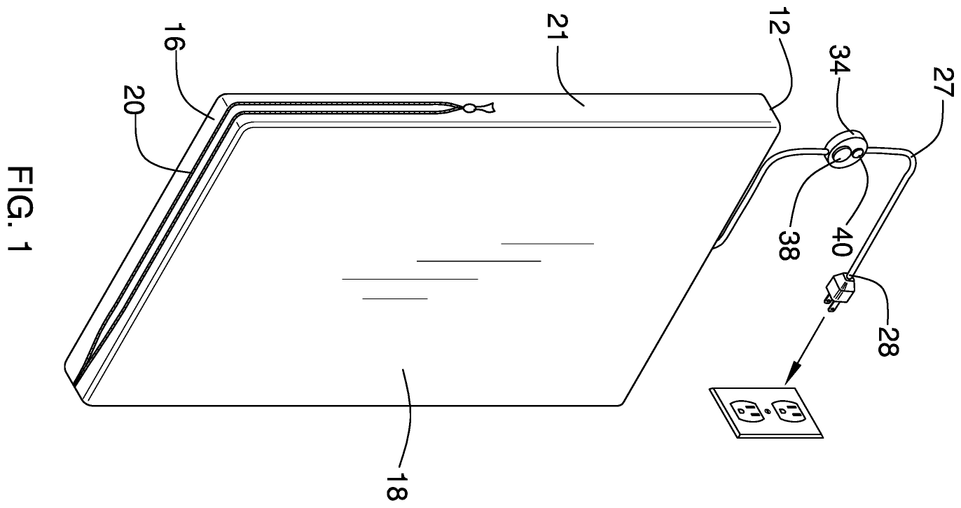

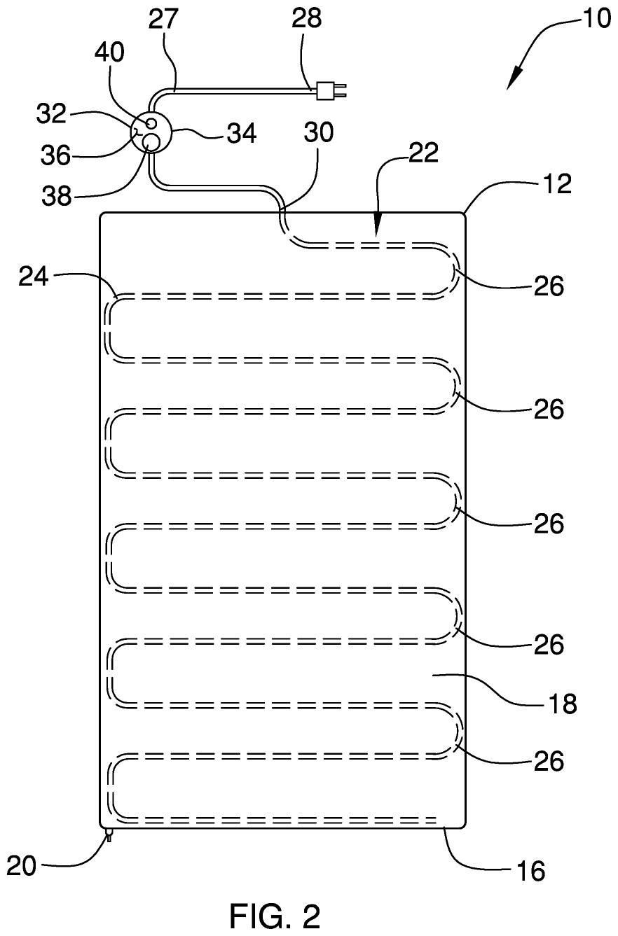

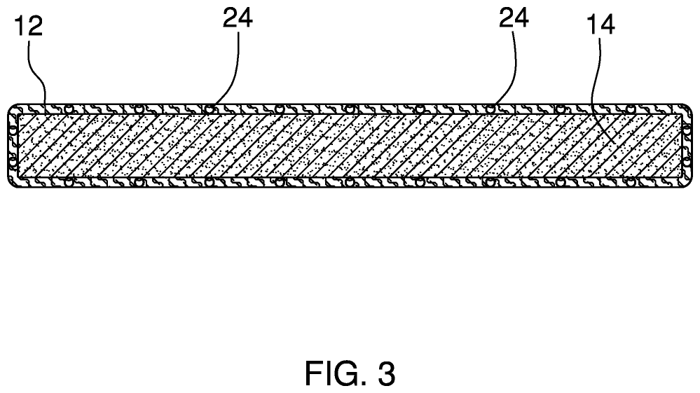

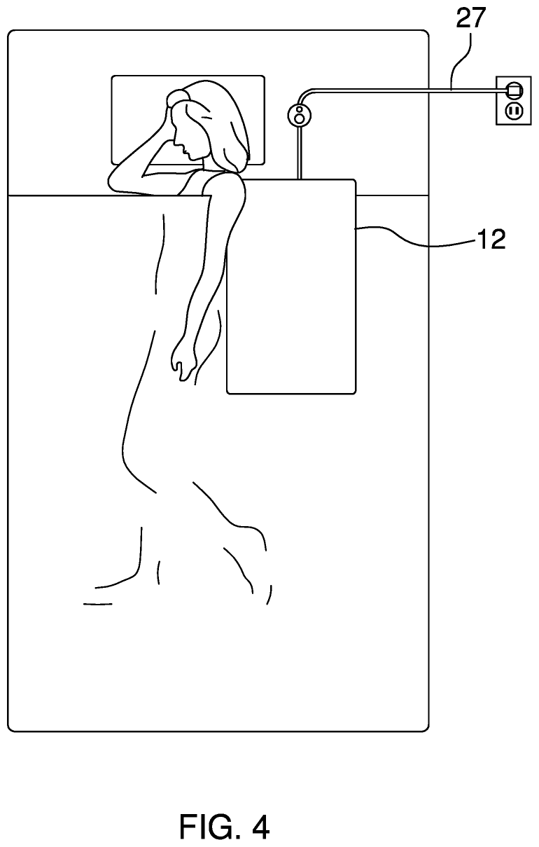

With reference now to the drawings, and in particular to FIGS. 1 through 6 thereof, a new pillowcase device embodying the principles and concepts of an embodiment of the disclosure and generally designated by the reference numeral 10 will be described.

As best illustrated in FIGS. 1 through 6, the heated pillowcase assembly 10 generally comprises a pillowcase 12 that has a length of at least 50.0 inches and a width of at least 20.0 inches. Thus, a body pillow 14 can be positioned within the pillowcase 12. The pillowcase 12 rests against a user when the user lies against the body pillow 14 while sleeping. The pillowcase 12 has a first end 16 and an outer wall 18, and the first end 16 is open to access an interior of the pillowcase 12 and insertably receive the body pillow 14. The pillowcase 12 may be comprised of a quilted material such as fleece or the like. Additionally, the pillowcase 12 may be comprised of a thermally conductive material.

A fastener 20 is coupled to the pillowcase 12 for opening and closing the pillowcase 12. The fastener 20 is positioned on and is coextensive with the first end 16 and additionally extends partially along a first lateral side 21 of the outer wall 18 of the pillowcase 12. The fastener 20 may comprise a zipper, a pair of hook and loop fasteners and any other type of linear fastener for opening and closing the pillowcase 12. Additionally, the fastener 20 may be comprised of a thermally insulating material, such as plastic or other material that resists thermal communication.

A heating unit 22 is coupled to the pillowcase 12 and the heating unit 22 is in thermal communication with the pillowcase 12. Thus, the heating unit 22 heats the pillowcase 12 when the heating unit 22 is turned on. In this way the heating unit 22 can keep the user warm when the user lies against the body pillow 14. Additionally, the heating unit 22 facilitates the user to be warmed without lying beneath a blanket thereby enhancing comfort for users who find sleeping beneath a blanket to be too warm.

The heating unit 22 comprises a heating element 24 that is positioned within the outer wall 18 of the pillowcase 12 such that the heating element 24 is in thermal communication with the outer wall 18. The heating element 24 is formed into a plurality of loops 26 that are distributed over an entirety of the outer wall 18. Additionally, the heating element 24 may be an electronic heating element 24 and may have an operational temperature ranging between approximately 80.0 degrees Fahrenheit and 100.0 degrees Fahrenheit.

A power cord 27 is coupled to and extends away from the outer wall 18 of the pillowcase 12 and the power cord 27 is electrically coupled to the heating element 24. The power cord 27 has a distal end 28 with respect to the pillowcase 12 and a plug is electrically coupled to the distal end 28 for plugging into a female electrical outlet. The heating element 24 may include an input port 30 and the power cord 27 may removably engage the input port 30 thereby facilitating the power cord 27 to be removed from the pillowcase 12 when the pillowcase 12 is laundered.

A control 32 is coupled to the power cord 27 and the control 32 turns the heating element 24 on and off. The control 32 comprises a housing 34 that is coupled to the power cord 27. The housing 34 has an outwardly facing surface 36 and the housing 34 is positioned between the pillowcase 12 and the plug on the power cord 27. A power button 38 is movably coupled to the outwardly facing surface 36 of the housing 34. The power button 38 is in electrical communication with the heating element 24 such that the power button 38 turns the heating element 24 on and off.

A heat increase button 40 is movably coupled to the outwardly facing surface 36 of the housing 34 and the heat increase button 40 is in electrical communication with the heating element 24. The heating element 24 is actuated to an increased operational temperature when the heat increase button 40 is manipulated. Moreover, the heating element 24 can be actuated to its maximum operational temperature with the heat increase button 40. A heat decrease button 42 is movably coupled to the outwardly facing surface 36 of the housing 34 and the heat decrease button 42 is in electrical communication with the heating element 24. The heating element 24 is actuated to a decreased operational temperature when the heat decrease button 42 is manipulated. Thus, the heating element 24 can be actuated to its minimum operational temperature with the heat decrease button 42.

In use, the body pillow 14 is positioned in the pillowcase 12 and the fastener 20 is manipulated to close the pillow case. The power button 38 is manipulated to turn the heating element 24 on when the user lies against the body pillow 14 or clutches the body pillow 14. In this way the user is kept warm for sleeping without lying beneath a blanket. Each of the heat increase 40 and heat decrease 42 buttons is manipulated to adjust the temperature of the heating element 24. The power cord 27 may be removed from the pillowcase 12 when the pillowcase 12 is being laundered thereby protecting the power button 38, the heat increase button 40 and the heat decrease button 42 from being damaged by water.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heated pillowcase assembly being configured to heat a user when the user is sleeping, said assembly comprising:
a pillowcase having a length of at least 50.0 inches and a width of at least 20.0 inches, said pillowcase having a body pillow being positioned therein wherein said pillowcase is configured to rest against a user when the user lies against the body pillow; and
a heating unit being coupled to said pillowcase, said heating unit being in thermal communication with said pillowcase such that said heating unit heats said pillowcase when said heating unit is turned on wherein said heating unit is configured to keep the user warm when the user lies against the body pillow.

2. The assembly according to claim 1, further comprising:
said pillowcase having a first end and an outer wall, said first end being open to access an interior of said pillowcase wherein said first end is configured to insertably receive the body pillow; and
a fastener being coupled to said pillowcase for opening and closing said pillowcase, said fastener being positioned on and being coextensive with said first end.

3. The assembly according to claim 1, wherein:
said pillowcase has an outer wall; and
said heating unit comprises a heating element being positioned within said outer wall of said pillowcase such that said heating element is in thermal communication with said outer wall, said heating element being formed into a plurality of loops being distributed over an entirety of said outer wall.

4. The assembly according to claim 3, further comprising a power cord being coupled to and extending away from said outer wall of said pillowcase, said power cord being electrically coupled to said heating element, said power cord having a distal end with respect to said pillowcase, said distal end having a plug being electrically coupled thereto for plugging into a female electrical outlet.

5. The assembly according to claim 4, further comprising a control being coupled to said power cord, said control turning said heating element on and off.

6. The assembly according to claim 5, wherein said control comprises a housing being coupled to said power cord, said housing having an outwardly facing surface.

7. The assembly according to claim 6, further comprising a power button being movably coupled to said outwardly facing surface of said housing, said power button being in electrical communication with said heating element such that said power button turns said heating element on and off.

8. The assembly according to claim 7, further comprising a heat increase button being movably coupled to said outwardly facing surface of said housing, said heat increase button being in electrical communication with said heating element, said heating element being actuated to an increased operational temperature when said heat increase button is manipulated.

9. The assembly according to claim 8, further comprising a heat decrease button being movably coupled to said outwardly facing surface of said housing, said heat decrease button being in electrical communication with said heating element, said heating element being actuated to a decreased operational temperature when said heat decrease button is manipulated.

10. A heated pillowcase assembly being configured to heat a user when the user is sleeping, said assembly comprising:
a pillowcase having a length of at least 50.0 inches and a width of at least 20.0 inches, said pillowcase having a body pillow being positioned therein wherein said pillowcase is configured to rest against a user when the user lies against the body pillow, said pillowcase having a first end and an outer wall, said first end being open to access an interior of said pillowcase wherein said first end is configured to insertably receive the body pillow;
a fastener being coupled to said pillowcase for opening and closing said pillowcase, said fastener being positioned on and being coextensive with said first end; and
a heating unit being coupled to said pillowcase, said heating unit being in thermal communication with said pillowcase such that said heating unit heats said pillowcase when said heating unit is turned on wherein said heating unit is configured to keep the user warm when the user lies against the body pillow, said heating unit comprising:
a heating element being positioned within said outer wall of said pillowcase such that said heating element is in thermal communication with said outer wall, said heating element being formed into a plurality of loops being distributed over an entirety of said outer wall;
a power cord being coupled to and extending away from said outer wall of said pillowcase, said power cord being electrically coupled to said heating element, said power cord having a distal end with respect to said pillowcase, said distal end having a plug being electrically coupled thereto for plugging into a female electrical outlet; and
a control being coupled to said power cord, said control turning said heating element on and off, said control comprising:
a housing being coupled to said power cord, said housing having an outwardly facing surface;
a power button being movably coupled to said outwardly facing surface of said housing, said power button being in electrical communication with said heating element such that said power button turns said heating element on and off;
a heat increase button being movably coupled to said outwardly facing surface of said housing, said heat increase button being in electrical communication with said heating element, said heating element being actuated to an increased operational temperature when said heat increase button is manipulated; and
a heat decrease button being movably coupled to said outwardly facing surface of said housing, said heat decrease button being in electrical communication with said heating element, said heating element being actuated to a decreased operational temperature when said heat decrease button is manipulated.
